(19) United States
(12) Patent Application Publication
(10) Pub. No.: US 2023/0111148 A1
(43) Pub. Date: Apr. 13, 2023

(54) EFFICIENT MULTI-DIMENSIONAL CARBON SOURCE AND METHOD FOR PREPARING THE SAME

(71) Applicant: Shenzhen Shenshui Water Resources Consulting Co., LTD., Shenzhen (CN)

(72) Inventors: Zifeng LU, Shenzhen (CN); Wei ZHANG, Shenzhen (CN); Guoyou HUO, Shenzhen (CN); Qingjie MENG, Shenzhen (CN); Qiuwei LI, Shenzhen (CN); Xu KANG, Shenzhen (CN); Qihua HUANG, Shenzhen (CN); Yuxia XIE, Shenzhen (CN); Lei SUN, Shenzhen (CN); Jiabao ZHANG, Shenzhen (CN); Cai JIANG, Shenzhen (CN); Yanan SI, Shenzhen (CN)

(21) Appl. No.: 17/852,940

(22) Filed: Jun. 29, 2022

(30) Foreign Application Priority Data
Oct. 11, 2021 (CN) 202111183711.3

(51) Int. Cl. C02F 3/28; C02F 11/04; C12N 1/20

(52) U.S. Cl. CPC C02F 3/28; C02F 11/04; C12N 1/20; C02F 2305/06; C02F 2101/16

(57) ABSTRACT

An efficient multi-dimensional carbon source and a method for preparing the same are disclosed. The efficient multi-dimensional carbon source includes the following components by weight: 30%-50% of alcohols, 20%-30% of biomass digestive liquid, 1%-10% of carbohydrates, 0.5% of polymethyl methacrylate and balance of water. A method for preparing the efficient multi-dimensional carbon source is further disclosed.

### CROSS-REFERENCE TO RELATED APPLICATION

This application claims the priority and benefit of Chinese patent application serial no. 202111183711.3, filed on Oct. 11, 2021. The entirety of Chinese patent application serial no. 202111183711.3 is hereby incorporated by reference herein and made a part of this specification.

### FIELD OF THE INVENTION

The present application relates to the technical field of carbon sources, in particular to an efficient multi-dimensional carbon source and a method thereof.

### DESCRIPTION OF RELATED ART

With the increasing economic development, people's living standards are improving day by day, and the amount of sewage discharged is also increasing day by day. However, due to the imperfect construction of the pipeline network and the low C/N of sewage, it is difficult for some sewage treatment plants to meet the new national standard for denitrification. At the same time, as a policy vigorously promoted by the government, the significance of resource regeneration has been gradually increased.

Studies have shown that, for producing one ton of biodiesel, 0.1 ton of by-product glycerol will be produced. It is reported that the global biodiesel production reached 29.1 million tons in 2015. Therefore, the recycling of crude glycerol as a by-product in the production process can effectively save resources. In addition to glycerol, crude glycerol also contains relatively high amount of inorganic salts, organic salts, pigments, trace catalysts, glycerides, etc., so it will be costly and complicated to purify glycerol by a purification technology. Crude glycerol contains higher amount of COD and has better biodegradability. Therefore, using it as an externally added carbon source in the field of sewage treatment will help to improve the comprehensive utilization of crude glycerol.

An anaerobic digestion process includes four steps: \textcircled{1} hydrolysis; \textcircled{2} acidification; \textcircled{3} hydrogen production; and \textcircled{4} methane production. A traditional anaerobic digestion process requires high temperature, stable neutral or alkaline environment, and long reaction time to provide a stable environment for the microorganisms in the reactor to produce methane. In the anaerobic digestion process for producing methane, VFAs generated by hydrolysis and acidification in the early stage of the reaction will accumulate in the reactor, which will lower the pH in the reaction system to acid (3-4), and the acidic conditions will inhibit the activity of methanogens, acidifying bacteria and reduce gas production. In the anaerobic digestion process, in order to inhibit the accumulation of VFAs, alkaline substances are often added to change the pH to promote the rapid reaction and conversion of the accumulated VFAs. However, the periodic addition of large amounts of alkaline substances will increase the production cost. At the same time, the reaction time of alkaline fermentation is long (8-20d). Therefore, in the anaerobic digestion process for producing methane, VFAs are not a target product, but an ideal option for externally added carbon source of sewage in the field of environmental protection. VFAs mainly includes acetic acid, propionic acid, butyric acid, etc., with higher denitrification rate and better biodegradability, so it is necessary to control the reaction conditions to stop the anaerobic digestion in the hydrolysis and acidification stage, so that the biomass digestive liquid contains a large amount of VFAs.

On the other hand, the carbon sources used in the field of environmental protection are mostly carbon sources such as sodium acetate, methanol, ethanol and glucose. Although sodium acetate has better effect as an externally added carbon source, the cost is relatively high, the sludge yield is high, and the dosage needs to be precisely controlled to avoid the effluent COD exceeding the standard. Methanol has certain biological toxicity, ethanol suffers from a safety risk during transportation, and glucose will cause the accumulation of nitrite in the reaction system.

Therefore, there is a need for developing a new technical solution to solve the above problems.

### BRIEF SUMMARY OF THE INVENTION

In view of the above, the present application provides an efficient multi-dimensional carbon source and a method for preparing the same, regarding to the defects and deficiencies in the prior art, which solves the difficulty in further improve the TN effluent standard in the current sewage treatment process due to the low COD of the influent water, and has the advantage of improving the denitrification effect of sewage treatment.

In some embodiments, the technical solution adopted in the present application is: an efficient multi-dimensional carbon source, including or consisting of the following components by weight: 30%-50% of alcohol, 20%-30% of biomass digestive liquid, 1%-10% of carbohydrate, 0.5%-1% of preservative and balance of water.

The present application further provides another technical solution: a method for preparing an efficient multi-dimensional carbon source, including the following steps:

Step I: weighting each raw material of formula with corresponding mass fractions;

Step II: successively adding the biomass digestive liquid, the alcohols, the carbohydrates, the water and the preservatives into a reactor, and mixing homogenously to obtain the efficient multi-dimensional carbon source.

In a further embodiment, a method for preparing the biomass digestive liquid includes the following steps:

Step a: collecting biomass;

Step b: transporting the collected biomass to a pretreatment unit for crushing and stirring to form a slurry;

Step c: feeding the slurry into an anaerobic free digestion reactor as a reaction matrix for the anaerobic free digestion reaction;

Step d: transporting the mud-water mixture obtained after the reaction is completed to a solid-liquid separator, then the supernatant and anaerobic sludge are obtained by separation, and the separated supernatant is the biomass digestive liquid.

In a further embodiment, in step a, after collecting the biomass, sorting out inorganic matters therein, then organic matters are left.

In a further embodiment, in step c, after the slurry is transported to the anaerobic free digestion reactor, the temperature is adjusted to between 25° C.-55° C., and no pH-adjusting agents are added.

In a further embodiment, in step d, the separated anaerobic sludge is returned to the anaerobic free digestion reactor through a return pipe.

In a further embodiment, the biomass is biomass waste, and the biomass waste includes at least one of fruit and vegetable waste, kitchen waste, sawdust and mushroom residue waste, starch waste water, dairy waste water, yellow slurry water and other wastes.

In a further embodiment, in step c, the time for carrying out the anaerobic free digestion reaction is controlled within three days.

In a further embodiment, the preservative is polymethyl methacrylate.

In a further embodiment, the alcohols are by-products produced from the production of biodiesel.

In above technical solution, the beneficial effects of the present application are:

1. In the present application, the efficient multi-dimensional carbon source includes the following components by weight: 30%-50% of alcohols, 20%-30% of biomass digestive liquid, 1%-10% of carbohydrates, 0.5%-1% of preservatives and balance of water, which has the advantages of, for example, low cost, fast reaction rate and high COD concentration. In particular, the raw material biomass of biomass digestive liquid is biomass waste, and alcohols are by-products produced by biodiesel production, so the cost is lower than that of chemical products such as sodium acetate.

2. In the present application, the efficient multi-dimensional carbon source uses biomass raw materials, so it contains more trace elements, nutrients, and growth factors for the growth of microorganisms in the activated sludge process to meet the metabolic needs, enhance the activity of microorganisms, and improve biomass in the reaction system.

3. In the present application, the efficient multi-dimensional carbon source has high COD concentration, so the amount added each time is small, thereby the volume of the carbon source stored each time can also be greatly reduced.

4. In the present application, the efficient multi-dimensional carbon source is prepared by above method, wherein the process is operated at room temperature and in a slightly acidic environment, so the energy consumption and cost of chemicals are reduced compared with the traditional anaerobic digestion process. When the pH is low, it is beneficial to produce lactic acid by lactobacillus, inhibit the activity of methanogens, and reduces gas production. In addition, the control to pH is based on the fact that the large quantities of VFAs produced in the acidification stage of anaerobic digestion can greatly reduce the pH in the reaction system. Therefore, the pH needs not to be adjusted by adding additional chemicals, which may lower the production costs.

5. In the present application, the efficient multi-dimensional carbon source is prepared by above method, which broadens recycling of by-products and wastes in various industries, and effectively improves the comprehensive utilization of by-products and wastes in various industries.

6. In the present application, the denitrification rate of the sewage treatment process is increased by 30%.

### BRIEF DESCRIPTION OF DRAWINGS

In order to explain the examples of the present application or the technical solutions in the prior art more clearly, Figures that need to be used in the description of the examples or the prior art is briefly introduced in the following. Obviously, the Figures in the following description are only some examples of the present application, and other Figures can also be obtained from these drawings for those ordinary skilled in the art without paying any creative effort.

FIG. 1 is a process flow diagram according to an embodiment of the present application.

### DETAILED DESCRIPTION

The present application will be further described in detail below with reference to the accompanying drawings.

The specific examples are only explanation to the present application, and do not intent to limit the present application. Modifications to the examples may be made by those skilled in the art as required within the protection scope of the Claims of the present application, without paying any creative work, after reading this specification, which are protected by patent law.

The present application provides an efficient multi-dimensional carbon source, consisting of the following components by weight: 30%-50% of alcohols, 20%-30% of biomass digestive liquid, 1%-10% of carbohydrates, 0.5%-1% of preservatives and balance of water. Its physical and chemical properties are as follows: pH=6.0-7.0, COD 600,000 mg/L.

In some embodiments, the preservative is polymethyl methacrylate.

The alcohols in the present application are by-products produced by the production of biodiesel.

The raw materials for the production of biomass digestive liquid in the above formula are produced by anaerobic free digestion technology. Biomass raw materials include but are not limited to: fruit and vegetable waste, kitchen waste, sawdust and mushroom residue waste, starch waste water, dairy waste water, yellow slurry water and other wastes.

The polymethyl methacrylate in the above formula functions to prolong the shelf life of the biomass digestive liquid.

To sum up, in the present application, by-products and wastes of various industries are used as raw materials to produce and compound the efficient multi-dimensional carbon source, which plays a positive role in recycling the wastes and improving the denitrification effect of sewage treatment.

Example 1: An efficient multi-dimensional carbon source, consisting of the following components by weight: 30% of alcohols, 30% of biomass digestive liquid, 10% of carbohydrates, 1% of polymethyl methacrylate and balance of water.

Example 2: an efficient multi-dimensional carbon source, consisting of the following components by weight: 50% of alcohols, 30% of biomass digestive liquid, 10% of carbohydrates, 1% of polymethyl methacrylate and balance of water.

Example 3: an efficient multi-dimensional carbon source, consisting of the following components by weight: 40% of alcohols, 25% of biomass digestive liquid, 5.5% of carbohydrates, 0.5% of polymethyl methacrylate and balance of water.

The efficient multi-dimensional carbon sources of the above three examples were utilized to sewage treatment, and the following data of results were obtained:

| Example 1 | | Example 2 | | Example 3 | |
|---|---|---|---|---|---|
| | 6 | | 6 | | 6 |
| | Nitrogen | | Nitrogen | | Nitrogen |
| C/N | Concentration | C/N | Concentration | C/N | Concentration |
| Time (t) | (mg/L) | Time (t) | (mg/L) | Time (t) | (mg/L) |
| 0 | 64.985 | 0 | 70.989 | 0 | 64.985 |
| 0.5 | 54.879 | 0.5 | 59.235 | 0.5 | 54.879 |
| 1 | 46.296 | 1 | 52.638 | 1 | 46.296 |
| 1.5 | 39.700 | 1.5 | 42.490 | 1.5 | 39.700 |
| 2 | 29.848 | 2 | 36.655 | 2 | 29.848 |
| 2.5 | 24.436 | 2.5 | 29.340 | 2.5 | 24.436 |
| 3 | 17.628 | 3 | 24.436 | 3 | 17.628 |
| 3.5 | 14.710 | 3.5 | 17.924 | 3.5 | 14.710 |
| 4 | 8.241 | 4 | 11.370 | 4 | 8.241 |
| 4.5 | 7.395 | 4.5 | 8.072 | 4.5 | 7.395 |
| 5 | 6.719 | 5 | 7.734 | 5 | 6.719 |
| Denitrification | 5.98 mg · g⁻¹ · h⁻¹ | Denitrification | 5.71 mg · g⁻¹ · h⁻¹ | Denitrification | 5.16 mg · g⁻¹ · h⁻¹ |
| Rate | | Rate | | Rate | |

### Conclusions:

1. It has the advantages of low cost, fast reaction rate and high COD concentration. Among them, the raw material biomass of biomass digestive liquid is biomass waste, and alcohols are by-products produced by biodiesel production, so the cost is lower than that of chemical products such as sodium acetate.

2. It contains more trace elements, nutrients, and growth factors for the growth of microorganisms in the activated sludge to meet the metabolic needs, enhance the activity of microorganisms, and increase the biomass in the reaction system.

3. The COD concentration is high, so the amount added each time is small, thereby the volume of carbon source stored each time can also be greatly reduced.

4. The denitrification rate of the sewage treatment process is increased by 30%.

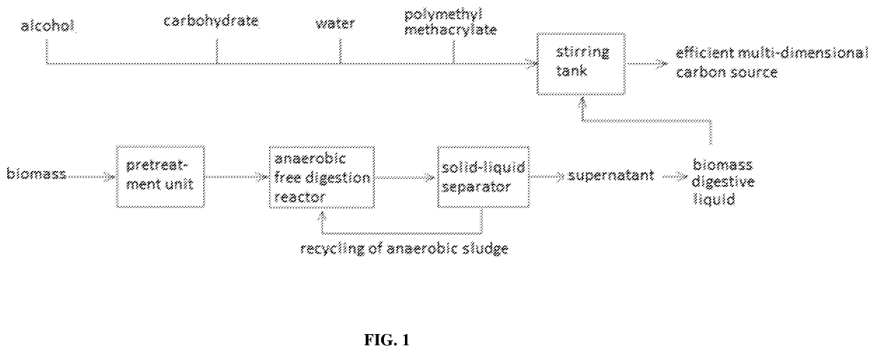

The present application also provides a method for preparing an efficient multi-dimensional carbon source, please refer to FIG. 1, including the following steps:

Step I: weighting each raw material of formula with corresponding mass fractions;

Step II: successively adding the biomass digestive liquid, the alcohols, the carbohydrates, the water and the polymethyl methacrylate into a reactor, and mixing homogenously to obtain the efficient multi-dimensional carbon source. In this example, the reactor was a stirring tank.

A method for preparing the biomass digestive liquid includes the following steps:

Step a: collecting biomass;

Step b: transporting the collected biomass to a pretreatment unit for crushing and stirring to form a slurry;

Step c: feeding the slurry into an anaerobic free digestion reactor as a reaction matrix for the anaerobic free digestion reaction;

Step d: transporting the mud-water mixture obtained after the reaction was completed to a solid-liquid separator, and the supernatant and anaerobic sludge were obtained by separation, and the separated supernatant was the biomass digestive liquid.

In particular, in step a, after collecting the biomass, sorting out inorganic matters therein, then the organic matter was left.

In particular, in step c, after the slurry was transported to the anaerobic free digestion reactor, the temperature is adjusted to between 25° C.-55° C., preferably 25° C., and no pH-adjusting agents were added. The digestive reaction produces various kinds of volatile fatty acids, lactic acid, etc. In the anaerobic free digestion process, with the progress of the reaction, the pH value in the system would gradually decrease to about 3 due to the large amount of organic acids produced. The activities of the methanogens and a part of acidifying bacteria were inhibited, while the activity of the lactobacillus was enhanced to produce a large amount of lactic acid. Without adding pH-adjusting agents, the pH in the system was maintained at about 3 until the reaction was completed.

In particular, in step d, the separated anaerobic sludge was returned to the anaerobic free digestion reactor through a return pipe to retain biomass and enhance the acid production effect.

The pH value selected for the process was 3-4; when the pH was low, it is beneficial to lactobacillus to produce lactic acid, the activity of methanogens was inhibited, and the gas production was reduced. In addition, the pH is controlled based on the fact that the large quantities of VFAs produced in the acidification stage of anaerobic digestion can greatly reduce the pH in the reaction system. Therefore, the pH need not to be adjusted by adding additional chemicals, which may lower the production costs.

The process reaction was controlled within 3 days. According to the previous test, when the reaction proceeded for 3 days, the anaerobic free digestion reaction has been completed by 80%, so the reaction time is selected for 3 days, which is conducive to speeding up the production.

The raw materials for the production of biomass digestive liquid in the above formula are produced using anaerobic free digestion technology. The specific method is as follows: a set of anaerobic free digestion reaction process is constructed by utilizing a characteristic that a large amount of VFAs produced in anaerobic digestion would greatly reduce pH in the reaction system, and combining the habits and characteristics of lactobacillus, so that the digestive liquid contains a large amount of lactic acid, VFAs and dissolved carbon source. The main purpose of anaerobic free digestion technology is to produce dissolved carbon source products (including proteins, VFAs, polysaccharides, lactic acid, etc.), which can be used as carbon sources for sewage feeding. This technology utilizes the acidifying bacteria in the reaction system to reduce the pH in the reaction system to about 3-4, inhibiting the activity of methanogens and part of acidifying bacteria, and activating the activity of lactobacillus, so as to achieve the purpose of producing large amount of lactic acid. During the anaerobic free digestion process, the rapidly generated volatile fatty acids will reduce the pH in the reactor system to 3-4. Methanogenic bacteria and part of acidifying bacteria are inhibited in this pH range, and the methane production rate and gas production rate are reduced. Therefore, soluble substances, lactic acid, a small amount of unhydrolyzed carbohydrates and proteins are mainly contained in the anaerobic free digestive liquid. These substances are all ideal options for externally added carbon source for sewage, which can effectively improve the denitrification effect of sewage. At the same time, natural raw materials are adopted, so that more trace elements are contained in the digestive liquid to meet the needs for microbial growth. Lactobacillus are cultivated in anaerobic free digestion process in an acidic environment to produce lactic acid in large quantities. Lactic acid has proved to be an ideal externally added carbon source. In this process, the reaction system is kept in an acidic environment without adjusting pH, which helps to reduce the yield of methane, reduces the loss of organic matrix, and also speeds up the reaction process, which can be shortened to 3-4 days. At the same time, a suitable temperature for anaerobic free digestion technology is around room temperature (25° C.), so this process does not require additional heating equipment or insulating equipment, thereby reducing energy consumption, costs and maintenance difficulty for production, compared with the anaerobic digestion process which needs to react at medium temperature (35° C.) or high temperature (55° C.),

Beneficial effects brought by the method for preparing the efficient multi-dimensional carbon source:

1. The energy consumption of process equipment and the costs of chemicals are low. The process is carried out at room temperature and a slightly acidic environment, so the energy consumption and the costs of chemicals are reduced, as well as the costs of chemicals for adjusting pH, compared with traditional anaerobic digestion processes.

2. Directions of recycling by-products and wastes in various industries are broaden. Using the carbon source and its production process, the extent for comprehensively utilizing by-products and wastes in various industries may be effectively improved.

3. The denitrification rate for sewage treatment process is improved by 30%.

4. The area for storing carbon sources is reduced. Due to high concentration of COD, the amount added each time is small, and the volume of carbon source stored each time may be greatly reduced as well.

The technical process of the present application is generally as follows: please refer to FIG. 1, transporting the biomass after being collected to a pretreatment unit for crushing and stirring to form a slurry, feeding the slurry into an anaerobic free digestion reactor as a reaction matrix, and transporting the mud-water mixture obtained after the reaction is completed to a solid-liquid separator, and the separated supernatant is the biomass digestive liquid. The separated anaerobic sludge is returned to the anaerobic free digestion reactor through a return pipe to reduce the loss of reaction microorganisms. Successively adding the biomass digestive liquid, alcohols, carbohydrates, water and polymethyl methacrylate into a stirring tank, and mixing homogenously to obtain the efficient multi-dimensional carbon source.

The above description are merely provided for an explanation to the technical solution of the present application, rather than imposing a limitation thereto. Other modifications or equivalent replacements to the technical solution of the present application made by those ordinary skilled in the art would fall within the scope defined by the claims of the present application, as long as they do not depart from the spirit and scope of the technical solution of the present application.

What is claimed is:

1. A carbon source, comprising the following components by weight: 30%-50% of alcohol, 20%-30% of biomass digestive liquid, 1%-10% of carbohydrate, 0.5%-1% of preservative, and balance of water.

2. A method for preparing the carbon source according to claim 1, comprising the following steps:
Step 1: weighting the components by weight; and
Step 2: adding the biomass digestive liquid, the alcohol, the carbohydrate, the water and the preservative into a reactor, and mixing homogenously to obtain the carbon source.

3. The method for preparing the carbon source according to claim 2, wherein the biomass digestive liquid is prepared by the following steps:
Step a: collecting a biomass;
Step b: crushing the biomass and forming a slurry;
Step c: subjecting the slurry to an anaerobic free digestion reaction in an anaerobic free digestion reactor; and
Step d: separating the slurry in a solid-liquid separator to obtain a supernatant and an anaerobic sludge, wherein the supernatant is the biomass digestive liquid.

4. The method for preparing the carbon source according to claim 3, wherein in step a, after collecting the biomass, organic matters in the biomass are maintained and inorganic matters in the biomass are discarded.

5. The method for preparing the carbon source according to claim 3, wherein in step c, a temperature in the anaerobic free digestion reactor is adjusted to between 25° C.-55° C., without adding a pH-adjusting agent.

6. The method for preparing the carbon source according to claim 3, wherein in step d, the anaerobic sludge is returned to the anaerobic free digestion reactor.

7. The method for preparing the carbon source according to claim 3, wherein the biomass is biomass waste, and the biomass waste comprises at least one selected from a group consisting of fruit and vegetable waste, kitchen waste, sawdust and mushroom residue waste, starch waste water, dairy waste water, and yellow slurry water.

8. The method for preparing the carbon source according to claim 3, wherein in step c, a time for the anaerobic free digestion reaction is not more than three days.

9. The method for preparing the carbon source according to claim 2, wherein the preservative is polymethyl methacrylate.

10. The method for preparing the carbon source according to claim 2, wherein the alcohol is a by-product produced in biodiesel production.
